(19) United States
(12) Patent Application Publication
(10) Pub. No.: US 2016/0331683 A1
(43) Pub. Date: Nov. 17, 2016

(54) WATER-IN-OIL TYPE EMULSION FOR TREATING A DISEASE OF THE EYE

(71) Applicant: SANTEN SAS, Evry (FR)

(72) Inventors: Frédéric LALLEMAN, Fresnes (FR); Jean-Sébastien GARRIGUE, Verrieres-le-Buisson (FR)

(21) Appl. No.: 15/193,508

(22) Filed: Jun. 27, 2016

Related U.S. Application Data
(62) Division of application No. 12/875,805, filed on Sep. 3, 2010.

(51) Int. Cl. A61K 9/107; A61K 47/14; A61K 31/573; C07K 16/22; A61K 38/17; A61K 47/22; A61K 9/00; A61K 47/26

(52) U.S. Cl. CPC A61K 9/107; A61K 9/0048; A61K 47/14; A61K 47/26; C07K 16/22; A61K 38/1709; A61K 47/22; A61K 31/573; C07K 2317/24; C07K 2317/55; C07K 2317/76; A61K 2039/505

(57) ABSTRACT

A composition is described herein for administering with a sustained release kinetic a therapeutically effective amount of a therapeutic agent to a subject in need thereof for treating diseases or conditions of the eye, wherein the composition is an water-in-oil type emulsion comprising an oil phase, a lipophilic surfactant dissolved in the oil phase, an aqueous phase dispersed in the oil phase, a hydrophilic therapeutic agent dissolved in the aqueous dispersed phase, and wherein the composition is intraocularly injectable, wherein the composition has a density lower than 1. Some embodiments also relate to a pharmaceutical composition or to a medicament comprising a composition described herein, and to a method for treating a condition or disease of the eye comprising administering a therapeutic amount of a composition described herein.

### FIELD

The embodiments relate to the field of the treatment of the conditions or diseases of the eye through the intraocular administration of therapeutic agents.

### BACKGROUND

The treatment of eye diseases by injecting a therapeutic agent directly in the vitreous chamber has shown promising results in the past. Macugen® (oligonucleotide) and Lucentis® (monoclonal antibody) are pharmaceutical products which are efficient to treat retinal diseases.

However, their half-life in the vitreous is relatively short leading to repeated injections to maintain the effect. The rapid clearance of these products is due to the renewal of the vitreous liquid over time.

This issue was already addressed in the prior art: for example, WO2009/046198 describes a method for administering a therapeutic agent in the vitreous with a sustained release kinetic; this method involves the formation of a macroscopic gel-like structure comprising said therapeutic agent, in the vitreous chamber. Also, EP2187980 describes the injection in the vitreous chamber of a therapeutic agent combined with a polymeric precursor, which will form in situ a hydrogel suitable for controlled release of said therapeutic agent.

However, the injection in the vitreous of a subject of a gel or gel-like structure as described in these patent applications may cause visual discomfort to the subject due to the invasion of the visual field by said gel or gel-like structure.

In another approach, a solid implant is injected in the eye of the subject, and the implant will release the active ingredient over several months. However, this form of administration may not be suitable for proteins and monoclonal antibodies.

Therefore, there remains a need for a method of providing sustained release in the vitreous chamber of a composition comprising a hydrophilic therapeutic agent, such as for example a protein or a nucleic acid. Ensuring the visual comfort of the patient when the composition within the vitreous body is another issue.

Surprisingly, the Applicant realized that a water-in-oil emulsion could be an efficient vehicle for administering hydrophilic therapeutic agents. Water-in-oil type emulsions are biphasic systems in which water droplets are dispersed within an oil phase.

The use of water-in-oil type emulsions as vehicles for sustained release of therapeutic agents is well known in the art. For example, WO01/89479 discloses the use of water-in-oil type emulsions for the parenteral administration of hydrophilic active ingredients with a sustained release kinetic. Chan el al. (Int. J. Pharm. 2007 Jan 2,328(1):65-71) specifically studied the use of water-in-oil type emulsions for topical delivery of an ocular drug with a sustained release kinetic. However, these prior art documents do not suggest the use of water-in-oil type emulsions for intraocular administration of a drug with a sustained release kinetic.

Some embodiments thus relate to the use of water-in-oil type emulsions for intraocular administration of a therapeutic agent to a subject in need thereof, providing a sustained release kinetic, and avoiding any invasion of the field of vision of the subject or safety issues.

An advantage of the solution proposed by the Applicant may be that some oil-in-water emulsions described herein may form a bubble having a lower density than the vitreous liquid. When injected, the bubble of the composition may slowly shift up from injection location to the upper part of the vitreous. Consequently, this liquid bubble may float over the vitreous, out of the visual field, avoiding any visual discomfort for the subject to which the composition is administered. Moreover, the composition may be in physical contact with both vitreous body and targeted tissues such as, for example, the choroid or the retina, and the release of the therapeutic agent may occur at the exact location of need.

### Definitions

As used herein, the following terms may have the following meanings:

“Emulsion”: includes a colloidal system made of two non-miscible elements, for example oil and water. One element (the dispersed phase) is present on the form of droplets dispersed in the other element, constituting the continuous phase.

“Water-in-oil type emulsion”: includes an emulsion made of water or aqueous droplets (i.e. the dispersed phase) dispersed in an oil phase (i.e. the continuous phase). A water-in-oil type emulsion also comprises surfactants (as defined hereafter), to avoid phase separation.

“Sustained release kinetic”: includes the slow release kinetic of a compound, at a predetermined rate and over an extended period of time.

“Intraocular administration”: includes injection of a product directly in the eyeball i.e. injection in the anterior chamber or in the posterior cavity (vitreous cavity) of the eye.

“Surfactant”: includes a substance that lowers the interfacial tension between two liquids.

“Bioresorbable”: includes a compound that progressively disappears in a biologic environment.

“Therapeutic agent”: includes a molecule or a substance_{;} preferably a biological molecule such as for example an oligonucleotide, a siRNA, a miRNA, a DNA fragment, an aptamer, an antibody and the like, or a chemical entity, having the capacity, when administered in a suitable amount, of slowing down or stopping the progression, aggravation, or deterioration of one or more symptoms of a disease, or condition; alleviates the symptoms of a disease or condition; cures a disease or condition.

“Therapeutically effective amount”: includes the amount of a therapeutic agent necessary and sufficient for slowing down or stopping the progression, aggravation, or deterioration of one or more symptoms of the disease, or condition; alleviating the symptoms of the disease or condition; curing the disease or condition.

“Hydrophilic”: includes a molecule or a portion of a molecule that is typically charge-polarized and capable of hydrogen bonding, enabling it to dissolve more readily in water than in oil or other solvents.

“Lipophilic”: includes a chemical compound capable to dissolve in fats, oils, lipids, and non-polar solvents.

“Non-miscible”: includes a liquid which does not combine or blend with another liquid, or which does not combine or blend immediately with another liquid.1

### SUMMARY

Some embodiments relate to a composition for administering with a sustained release kinetic a therapeutically effective amount of a therapeutic agent to a subject in need thereof for treating diseases or conditions of the eye, wherein the composition is a water-in-oil type emulsion comprising an oil phase, a lipophilic surfactant dissolved in the oil phase, an aqueous phase dispersed in the oil phase, a hydrophilic therapeutic agent dissolved in the aqueous dispersed phase, wherein the composition is intraocularly injectable, and wherein the composition has a density lower than 1.

According to an embodiment, the oil phase is selected from the group comprising triglycerides such as, for example, medium chain or long chain triglycerides, monoglycerides, diglycerides, vegetable oils or mineral oils .

Preferably, the lipophilic surfactant comprises a sorbitan ester such as, for example, sorbitan stearate, sorbitan laurate and sorbitan monopalmitate, bentonite, glycerol monostearate and propylene glycol monolaurate or mixtures thereof.

In a preferred embodiment, the aqueous phase is present in the composition in an amount ranging from 0.1 to less than 50% in weight to the total weight of the composition, preferably from 0.5 to 15% w/w, more preferably from 2 to 10% w/w. Preferably, the hydrophilic therapeutic agent is selected from monoclonal antibodies (full or fragment Fab), such as for example ranibizumab; anti-angiogenic or anti-complement molecules, such as for example anginex or lodamin; a ROCK (Rho-kinases) inhibitor, such as for example fasudil; tetrapyridoether against dry age macular degeneration; small peptides such as for example anti-B1 peptide R-954 to proteins such as anti-CD160 S-HLA-G; enzymes such as for example superoxide dismutase or catalase; WNT3A protein which activates WNT (Wingless-Integration site) for survival of photoreceptor cells; growth factors such as epithelium growth factors (EGF), anti-EGF or TGF (Transforming growth factor); siRNA such as siRNA anti-arginase, miRNA; oligonucleotides such as antisens DNA or antisens RNA; antioxidant small molecules such as EUK (Eukaryon) family, for example EUK-143 sodium catalase mimetic; iron chelating molecules such as deferiprone and salicylaldehyde isonicotinoyl hydrazone; anti-inflammatory molecules such as epigallocatechin gallate; free radical scavengers such as edaravone; or antibiotics for back of the eye infection such as linezolide, anti-inflammatory molecules preferably selected from the group comprising lipophilic cyclosporine A, dexamethasone and its hydrophilic derivatives, or mixtures thereof.

In one embodiment, the composition further comprises a lipophilic therapeutic agent in the oil phase, said lipophilic therapeutic agent being selected from lutein, alpha-tocopherol and dexamethasone-palmitate.

The composition may further comprise viscosity modifying agents, such as, for example an hydrogel, and/or pH buffering agents, such as, for example, phosphate, citrate, tris, histidine or acetate buffer, and/or osmolality modifying agents, such as, for example NaCl, KCl, CaCl₂, glycerol, mannitol, alpha-trehalose or propylene glycol.

In some embodiments, the composition is intravitreally injectable.

The diseases or conditions of the eye that may be treated with the compositions described herein are selected from the group comprising glaucoma, anterior uveitis retinal oxidation, age related macular degeneration, posterior uveitis, diabetic macular edema and central vein occlusion.

Some embodiments also relates to a pharmaceutical composition comprising a water-in-oil type emulsion described herein, and further comprising one or more pharmaceutically acceptable excipients.

Some embodiments also relate to a medicament comprising a water-in-oil type emulsion as described above.

Some embodiments also relate to a device comprising the composition or the medicament described herein. According to a preferred embodiment, the composition, the pharmaceutical composition, the medicament or the device are not implants.

Some embodiments also relate to a method for treating a condition or disease of the eye, wherein a volume of 5 to 250 microliters of the composition or the medicament is injected in the vitreous chamber or in the anterior chamber. According to some methods, the injected composition forms in situ a bubble within which the aqueous phase migrates towards the surface of a bubble, for sustained release of the therapeutic agent to the vitreous chamber, to the anterior chamber or the targeted tissue.

### DETAILED DESCRIPTION

Some embodiments thus relate to a composition for administering with a sustained release kinetic a therapeutically effective amount of a therapeutic agent to a subject in need thereof for treating diseases or conditions of the eye, wherein the composition is a water-in-oil type emulsion comprising an oil phase, a lipophilic surfactant dissolved in the oil phase, an aqueous phase dispersed in the oil phase and a hydrophilic therapeutic agent dissolved in the aqueous dispersed phase, the composition being intraocularly injectable and having a density lower than 1.

Due to the fact that its continuous phase is essentially composed of oil, the water-in-oil type emulsion may present a lower density than the vitreous liquid which has a density equivalent, if not equal, to the density of water. Thus, in some embodiments, the density of the water-in-oil type emulsion is less than 1. Preferably, the density of the water-in-oil type emulsion ranges from 0.9 to 0.99, more preferably from 0.94 to 0.98. Therefore, when injected, the solution may form a liquid, non breakable and non miscible bubble. When injected in the vitreous body, the bubble will be located over the vitreous liquid.

In one embodiment, the emulsion further comprises one or more lipophilic surfactants, in an amount sufficient for ensuring the water-in-oil type of the emulsion. In a particular embodiment, said lipophilic surfactants are selected from sorbitan ester such as, for example, sorbitan stearate, sorbitan laurate, and sorbitan monopalmitate, bentonite, glycerol monostearate and propylene glycol monolaurate or mixtures thereof.

In a particular embodiment, the HLB (hydrophilic-lipophilic Balance) of the surfactants of the composition ranges from 0 to 9, preferably from 2 to 8.

In a particular embodiment, the amount of lipophilic surfactants in the water-in-oil type emulsion ranges from 0.1 to 10% in weight to the weight of the total emulsion, preferably from 0.5 to 5% w/w, more preferably from 1 to 2% w/w.

In one embodiment, the aqueous phase in the water-in-oil type emulsion is present in an amount ranging from 0.1 to less than 50% in weight to the weight of the total emulsion, preferably from 0.5 to 15% w/w, more preferably from 2 to 10% w/w. Preferably, said aqueous phase is water or is essentially composed water.

In a particular embodiment, the composition includes one or more hydrophilic therapeutic agent(s) present in the aqueous droplets of the water-in-oil type emulsion.

In one embodiment, the hydrophilic therapeutic agent is selected from the group comprising monoclonal antibodies (full or fragment Fab), such as for example ranibizumab; anti-angiogenic or anti-complement molecules, such as for example anginex or lodamin; a ROCK (Rho-kinases) inhibitor, such as for example fasudil; tetrapyridoether against dry age macular degeneration; small peptides such as for example anti-B1 peptide R-954 to proteins such as anti-CD160 S-HLA-G; enzymes such as for example superoxide dismutase or catalase; WNT3A protein which activates WNT (Wingless-Integration site) for survival of photoreceptor cells; growth factors such as epithelium growth factors (EGF), anti-EGF or TGF (Transforming growth factor); siRNA such as siRNA anti-arginase, miRNA; oligonucleotides such as antisens DNA or antisens RNA; antioxidant small molecules such as EUK (Eukaryon) family, for example EUK-143 sodium catalase mimetic; iron chelating molecules such as deferiprone and salicylaldehyde isonicotinoyl hydrazone; anti-inflammatory molecules such as epigallocatechin gallate; free radical scavengers such as edaravone; or antibiotics for back of the eye infection such as linezolide, anti-inflammatory molecules preferably selected from the group comprising lipophilic cyclosporine A, dexamethasone and its hydrophilic derivatives and mixtures thereof.

In an embodiment, the amount of hydrophilic therapeutic ingredient in the emulsion ranges from 0.01 to 10% in weight to the total weight of the emulsion, preferably from 0.05 to 5% w/w, more preferably from 0.1 to 1% w/w.

In an embodiment, the emulsion further comprises one or more lipophilic therapeutic agents in the oil phase. In a preferred embodiment, said lipophilic therapeutic agent is selected from lutein, alpha-tocopherol and dexamethasone-palmitate.

In a preferred embodiment, the amount of hydrophilic therapeutic ingredient in the emulsion ranges from 0.01 to 10% in weight to the total weight of the emulsion, preferably from 0.05 to 5% w/w, more preferably from 1 to 2% w/w.

The water-in-oil type emulsion may be effective for sustained release administration of a therapeutic agent. Said sustained release effect is provided by the migration of water droplets dispersed in the continuous oil phase to the surface of the oil bubble formed by the emulsion when injected in the eye. In one embodiment, the sustained release kinetic can be adapted to the exact need of the patient.

In a first embodiment, said sustained release kinetic may depend on the physico-chemical properties of the oil phase. The more viscous the oil phase is, the more extended the period of release may be. With viscous oil such as long chain triglycerides, the release may be extended up to one year. In one embodiment, the oil phase of the water-in-oil type emulsion comprises an oil selected from the group comprising triglycerides such as, for example semi-synthetic oils: medium chain triglycerides (MCT) or long chain triglycerides; monoglycerides, diglycerides or vegetable oils such as, for example, castor oil or mineral oils. According to an embodiment, the viscosity of the oil phase ranges from 1 to 10000 mPa.s at 20° C., preferably from 10 to 5000 mPa.s at 20° C., even more preferably from 25 to 1000 mPa.s at 20° C.

In a second embodiment, said sustained release kinetic may depend on the size of the water droplets dispersed in the oil phase. The smaller the droplets are, the longer their migration to the surface of the injected bubble may be, and then the more extended the period of release may be. For example, for comparable compositions in terms of ingredients, an emulsion with a droplet size of more than 1 nm may release the therapeutic agent in about 1 week to 2 months, whereas the release may be increased to more than 2 months when the droplet size is below 500 nm.

In a third embodiment, said sustained release kinetic may be conditioned by the volume of the injected water-in-oil type emulsion. The bigger the emulsion bubble is, the more extended the period of release may be. Preferably, a volume of the composition ranging from 5 to 250 μL, preferably from 10 to 100 μL, more preferably about 50 μL is injected.

In a fourth embodiment, the viscosity of the aqueous phase is increased in order to enhance the sustained release. In a particular embodiment, said viscosity is increased by addition of a hydrogel. In a preferred embodiment, said hydrogel is made of cellulose, hyaluronic acid, and/or collagen.

In a fifth embodiment, the means for sustained release of the therapeutic agents as described in the first to four embodiments hereabove, may be combined one to each other or all together in order to modulate the sustain release effect.

According to an embodiment, the aqueous phase of the emulsion further comprises a pH modifying agent or a pH buffering agent. In a preferred embodiment, said pH buffering agent is selected from the group comprising phosphate, citrate, tris, histidine or acetate buffers. In a preferred embodiment, said pH buffering agent is a phosphate buffer. In one embodiment, the amount of said agent for modifying the pH of the aqueous phase ranges from 0.05 to 10% in weight to the total weight of the aqueous phase, preferably from 0.01 to 5% w/w, more preferably from 0.1 to 1% w/w.

According to an embodiment, the aqueous phase of the emulsion further comprises an agent for modifying the osmolality of the aqueous phase of the emulsion. In a first embodiment, said agent for modifying the osmolality is selected from the group comprising NaCl, KCl and CaCl₂. In a second embodiment, the modification of the osmolality of the composition results from the addition of a compound selected from the group comprising neutral compounds such as, but not limited to, glycerol, mannitol, alpha-trehalose or propylene glycol. In a preferred embodiment, the modification of the osmolality of the composition results from the addition of 0.5-2%, preferably 0.9% w/w of NaCl, 0.51 0%, preferably 3-5% w/w of alpha-trehalose or mannitol or propylene glycol in weight to the weight of the total emulsion.

In one embodiment, if the water-in-oil type emulsion is too viscous to be injected, the emulsion can be re-emulsified into a water phase to form a multiple emulsion of the type water-in-oil-in-water.

According to an embodiment, the composition is intraocularly injectable. Preferably, the composition is intravitreally injectable.

The water-in-oil type emulsion is bioresorbable. In one embodiment, the oily bubble is resorbed in a period of time ranging from 1 to 24 months after injection, preferably from 6 to 18 months after injection, more preferably about 12 months after injection.

The water-in-oil type emulsions described herein may be for treating diseases or conditions of the eye. In one embodiment, said diseases or conditions of the eye are selected from the group comprising glaucoma, anterior uveitis retinal oxidation, age related macular degeneration, posterior uveitis, diabetic macular edema and central vein occlusion.

Some embodiments also relate to a pharmaceutical composition according to a water-in-oil type emulsion described herein. In one embodiment, the pharmaceutical. composition further comprises at least one pharmaceutically acceptable excipient.

Some embodiments also relate to a medicament according to the water-in-oil type emulsion described herein.

Some embodiments also relate to a device for administering the water-in-oil type emulsion, the pharmaceutical composition or the medicament described herein. Preferably, said device is a prefilled syringe. In one embodiment, said device contains the pharmaceutical composition or the medicament described herein.

Also, the some embodiments also relate to a method for treating a condition or disease of the eye, comprising administering intraocularly a therapeutic amount of the composition or of the medicament. Preferably, the method comprises the injection, preferably in the vitreous chamber, of a volume ranging from 5 to 250 μL, preferably from 10 to 100 μL, more preferably of about 50 μL. In a preferred embodiment, said composition or medicament is injected less than once a week, preferably less than once a month, more preferably less than once in six months. According to an embodiment, the injected. composition forms in situ a bubble within which the aqueous phase migrates towards the surface of a bubble, providing sustained release of the therapeutic agent to the vitreous chamber or the targeted tissue.

Some embodiments are further illustrated by the following examples.

### EXAMPLES

### Example 1: Composition Comprising Ranibizumab

| Ingredients | Concentration |
|---|---|
| ranibizumab | 0.1% |
| Water for injection | 4% |
| dihydrated alpha, alpha-trehalose | 3% |
| Monohydrated histidine chlorhydrate histidine | 0.05% |
| Sorbitan stearate | 2% |
| Medium chain triglyceride | Qs 100% |

### Example 2: Composition Comprising R-954

| Ingredients | Concentration |
|---|---|
| Peptide R-954 | 0.8% |
| Water for injection | 5% |
| Glycerol monostearate | 0.5% |
| Sorbitan monoplamitate | 1% |
| Medium chain triglyceride | Qs 100% |
| Dexamethasone palmitate | 1.2% |

1-18. (canceled)

19. A method for treating a condition or disease of the eye comprising administering to a subject in need thereof by intraocular injection a therapeutic amount of a composition wherein the composition is a water-in-oil type emulsion comprising:
an oil phase;
a lipophilic surfactant dissolved in the oil phase;
an aqueous phase dispersed in the oil phase; and
said hydrophilic therapeutic agent dissolved in the aqueous dispersed phase;
wherein the composition is formulated for intraocular injection;
wherein the composition has a density lower than the density of water;
wherein said oil phase has a viscosity ranging from 1 to 10000 mPa.s at 20° C.

20. (canceled)

21. The method of claim 19, wherein the oil phase comprises a triglyceride, a monoglyceride, a diglyceride, a vegetable oil or a mineral oil.

22. The method of claim 21, wherein the triglyceride comprises a medium chain triglyceride or a long chain triglyceride.

23. The method of claim 19, wherein the lipophilic surfactant comprises a sorbitan ester, bentonite, glycerol monostearate and propylene glycol monolaurate and mixtures thereof.

24. The method of claim 23, wherein the sorbitan ester comprises sorbitan stearate, sorbitan laurate and sorbitan monopalmitate.

25. The method of claim 19, wherein the aqueous phase is present in an amount ranging from 0.1% by weight to less than about 50% by weight to the total weight of the composition.

26. The method of claim 25, wherein the aqueous phase is present in an amount ranging from about 0.5% by weight to about 15% w/w by weight to the total weight of the composition.

27. The method of claim 26, wherein the aqueous phase is present in an amount ranging from about 2% by weight to about 10% by weight to the total weight of the composition.

28. The method of claim 19, wherein said hydrophilic therapeutic agent comprises a monoclonal antibody, an anti-angiogenic or anti-complement molecule, a Rho-kinase inhibitor, a tetrapyridoether for treating dry eye related macular degeneration, a small peptide, an enzyme, a WNT3A protein which activates Wingless-Integration site for survival of photoreceptor cells, a growth factor, siRNA, miRNA, an oligonucleotides, an anti oxidant small molecule, an iron chelating molecule, an anti-inflammatory molecule, a free radical scavenger, or an antibiotic for back of the eye infection, or mixtures thereof.

29. The method of claim 19, wherein said hydrophilic therapeutic agent comprises a monoclonal antibody, a full or fragment Fab, ranibizumab, an anti-angiogenic molecule, an anti-complement molecule, anginex, lodamin, a Rho-kinase inhibitor, fasudil, a tetrapyridoether for treating dry age related macular degeneration, a small peptide, anti-B1 peptide R954, anti-CD160S-HLA-G, enzymes, superoxide dismutase or catalase, a WNT3A protein which activates Wingless-Integration site for survival of photoreceptor cells, a growth factor, an epithelium growth factors (EGF), anti-EGF, anti Transforming growth factor, siRNA, siRNA anti-arginase, miRNA, antisens DNA, antisens RNA, antioxidant small molecules, a Eukaryon family molecule, EUK-143 sodium catalase mimetic, an iron chelating molecule, deferiprone, salicylaldehyde isonicotinoyl hydrazine, an anti-inflammatory molecule, epigallocatechin gallate, free radical scavenger, edaravone, an antibiotics for back of the eye infection, linezolide, cyclosporine A, dexamethasone, hydrophilic derivatives of dexamethasone, or mixtures thereof.

30. The method of claim 19, wherein the composition further comprises a lipophilic therapeutic agent in the oil phase, wherein said lipophilic therapeutic agent comprises lutein, alpha-tocopherol or dexamethasone palmitate.

31. The method of claim 19, wherein the composition further comprises a viscosity modifying agent, a pH buffering agent, an osmolality modifying agent or a combination thereof.

32. The method of claim 19, wherein the composition further comprises a hydrogel, phosphate, citrate, tris, histidine, acetate buffer, NaCl, KCl, CaCl₂, glycerol, mannitol, alpha-trehalose, propylene glycol, or a combination thereof.

33. The method of claim 19, wherein the composition is injected intravitreally.

34. The method of claim 19, wherein the condition or disease of the eye to be treated is selected from the group comprising glaucoma, anterior uveitis retinal oxidation, age related macular degeneration, posterior uveitis, diabetic macular edema and central vein occlusion.

35. The method of claim 19, wherein the composition further comprises one or more pharmaceutically acceptable excipients.

36. The method of claim 19, wherein the composition is injected in the form of a medicament comprising said composition.

37. The method of claim 19, wherein a volume of 5 to 250 microliters of the composition or of a medicament comprising said composition is injected in the vitreous chamber or anterior chamber.

38. The method of claim 19, wherein the injected composition provides sustained release of the therapeutic agent to the vitreous chamber, to the anterior chamber or to a targeted tissue.

39. A method for administering with a sustained release kinetic a therapeutically effective amount of a hydrophilic therapeutic agent to a subject in need thereof, comprising administrating by intraocular injection a composition being a water-in-oil type emulsion comprising: an oil phase;
a lipophilic surfactant dissolved in the oil phase;
an aqueous phase dispersed in the oil phase; and
said hydrophilic therapeutic agent dissolved in the aqueous dispersed phase;
wherein the composition is formulated for intraocular injection;
wherein the composition has a density lower than the density of water;
wherein said oil phase has a viscosity ranging from 1 to 10000 mPa.s at 20° C.

40. The method of claim 39, wherein the method further comprises treating a condition or disease of the eye in the subject, wherein the condition or disease is treated by administering by intraocular injection with a sustained release kinetic, a therapeutically effective amount of the composition comprising the hydrophilic therapeutic agent to the subject.
